(19) United States
(12) Patent Application Publication
(10) Pub. No.: US 2026/0305604 A1
(43) Pub. Date: Oct. 8, 2026

(54) AN ORGAN CONTAINER, AN IMPEDANCE METER FOR THE ORGAN CONTAINER, AND A METHOD FOR DEFINING A DETERIORATION STATE OF AN ORGAN

(71) Applicant: TAMPERE UNIVERSITY FOUNDATION SR, Tampereen yliopisto (FI)

(72) Inventors: Jarno TANSKANEN, Tampere (FI); Jari HYTTINEN, Tampere (FI)

(21) Appl. No.: 19/483,257

(22) PCT Filed: May 17, 2024
(86) PCT No.: PCT/EP2024/063790

§ 371 Date: Nov. 11, 2025

(30) Foreign Application Priority Data
Jun. 9, 2023 (FI) 20235651

(51) Int. Cl. A01N 1/146; G01N 27/02; G01N 33/483

(52) U.S. Cl. CPC A01N 1/146; G01N 27/028; G01N 33/4833

(57) ABSTRACT

An organ container (1) comprises a wall (10) surrounding an interior space (11) configured to receive an organ (13). An electric conductor (14) and an impedance meter (16) are provided to measure a first impedance value at a first period of time and a second impedance value at a second period of time. Based on the difference between the first impedance and the second impedance, a deterioration state of the organ residing in the interior space is defined. In one example the electric conductor forms at least one loop around the interior space (11). In another example the electric conductor forms a single loop on a single side of a wall or at a bottom of the organ container. Multiple loops of multiple electric conductors may be provided, wherein each conductor may provide individual impedance measurements. In another example, the electric conductor is arranged as an individual impedance measurement device that may be placed inside the organ container.

### BACKGROUND

The present disclosure relates to physical analysis of biological material, and more particularly to transporting organs viable for successful implantation into a human or other mammal recipient.

Organ transplants often require transporting the organ after donation, as the donors and the donor hospitals may be located far from the transplantation hospital. The viability of the organ must be protected during the transport, all incidents and serious adverse events affecting the quality and safety of organs should be registered. Many organs may be classified as marginal and rejected after the transport, therefore quality assessment and prediction of the organ viability function is of utmost importance. For organ transplants, the transplanting surgeon must make the decision on whether the organ is acceptable for transplantation based on limited amount of information, such as the donor information, transplant diagnostic information at harvest time and the duration of the transportation.

It is estimated that over 140,000 organ transplantations were performed globally in 2018. For example, in 2018 in the USA, 3755 kidneys were discarded, which amount to 17.9 % of the donated kidneys. Further rejected organs included 278 pancreata, 707 livers, 3 intestines, 23 hearts, and 317 lungs. Some of these organs might have been viable but were rejected due to uncertainties during the transport. There is a great and increasing need for more donated organs and salvaging as many harvested organs as possible.

The organ transplant policies are well documented and maintained, for example by OPTN (Organ Procurement and Transplantation Network) in the USA. In Finland, a set of policies is listed in the document “National action plan for organ donation and transplantation 2023-2033”. Each organ transplant must follow these strict policies.

### SUMMARY

This summary is provided to introduce a selection of concepts in a simplified form that are further described below in the detailed description. This summary is not intended to identify key features or essential features of the claimed subject matter, nor is it intended to be used to limit the scope of the claimed subject matter. Furthermore, the claimed subject matter is not limited to implementations that solve any or all disadvantages noted in any part of this disclosure.

An organ transplant or other transportable tissue is repeatedly measured during a transport. An organ container comprises an electric conductor configured to form a coil that produces an electric field that extends to the organ transplant inside the organ container. In one example the electric conductor forms a loop or a coil around the organ container, surrounding the space for the organ transplant. In one example the electric conductor forms a single loop on a single side of a wall or at a bottom of the organ container. In one example the organ container wall comprises multiple loops of multiple electric conductors, wherein each conductor may provide individual impedance measurements.

In one embodiment, the electric conductor is arranged as an individual impedance measurement device that may be placed inside the organ container.

An impedance measured from the electric conductor has one component related to the organ transplant conductance. The organ transplant conductance is proportional to the organ transplant deterioration. The organ transplant deterioration may be due to biological tissue degradation at any level, including at molecular, cellular, organ structure and organ levels, physical trauma, such as mechanical or temperature induced damage, lack of oxygen or other substances, viruses, other pathogens or parasites, chemical insults, including alcohol consumption, cancer, surgical incidents, or other tissue damaging effects.

When the organ transplant is placed into the organ container, a first impedance measurement is made to with the organ transplant. The impedance measurement may be repeated periodically to monitor possible degradation of the organ transplant. The impedance measurement may be run through multiple frequencies. The deterioration state or the deterioration rate of the organ may be used to assess viability of the organ.

The impedance measurement does not require direct contact to the organ transplant. The electric conductor may be placed inside the organ container, where it does not affect the organ transplant. The measurements may be autonomous, without any involvement from the transport personnel. Current organ transplant policies may be applied.

In one example, the organ transplant should be placed inside three sterile bags, wherein each of the bags may be regarded as individual organ containers. The impedance measurement coil may be implemented outside the innermost sterile bag, for example to the second sterile bag.

The invention solves the problem of assessing the status of a harvested organ, by detecting changes in the deterioration state. Tissue deterioration state assessment can be performed continuously during transport and in real-time in the transplantation operating room. This provides the transplanting surgeon with contactless measurement data regarding the organ/tissue status and possible deterioration state during transport. The transplanting surgeon has a new tool and further data to decide on the organ status and whether or not to continue with the transplantation surgery. Transplant diagnostics is a time-consuming process at the recipient hospital, which could be mitigated with the help of data gathered during the transport.

Many of the attendant features will be more readily appreciated as they become better understood by reference to the following detailed description considered in connection with the accompanying drawings. The embodiments described below are not limited to implementations which solve any or all the disadvantages of donor organ transportation.

### BRIEF DESCRIPTION OF THE DRAWINGS

The present description will be better understood from the following detailed description read in light of the accompanying drawings, wherein:

FIG. 1 illustrates schematically one exemplary embodiment of an organ container;

FIG. 2 illustrates schematically a simulated graph of a relation of a measured impedance and the conductance of a deteriorating organ;

FIG. 3 illustrates three exemplary plots of an impedance measurement:

FIG. 4a illustrates schematically one exemplary frequency spectrum from the organ container without significant organ deterioration;

FIG. 4b illustrates schematically one exemplary frequency spectrum from the organ container with organ deterioration showing at some frequencies;

FIG. 5 illustrates schematically one exemplary embodiment of the organ container;

FIG. 6 illustrates schematically one exemplary embodiment of the organ container having non-rigid walls;

FIG. 7a illustrates schematically one exemplary system for transporting organs by the organ container;

FIG. 7b illustrates schematically one exemplary system for transporting organs by the organ container;

FIG. 8 illustrates a flowchart of steps of a method for defining a deterioration state of the organ;

FIG. 9 illustrates schematically one exemplary embodiment of an impedance measurement device;

FIG. 10a illustrates schematically one exemplary embodiment of the organ container from a first viewing angle;

FIG. 10b illustrates schematically one exemplary embodiment of the organ container from a second viewing angle; and

FIG. 11 illustrates schematically one exemplary system for transporting organs by the organ container.

Like reference numerals are used to designate like parts in the accompanying drawings.

### DETAILED DESCRIPTION

The detailed description provided below in connection with the accompanying drawings is intended as a description of the present examples and is not intended to represent the only forms in which the present example may be constructed or utilized. However, the same or equivalent functions and sequences may be accomplished by different examples.

Although the present examples are described and illustrated herein as being implemented in an organ container, the device or the method described are provided as an example and not a limitation. As those skilled in the art will appreciate, the present examples are suitable for application in a variety of different types of tissue transportation.

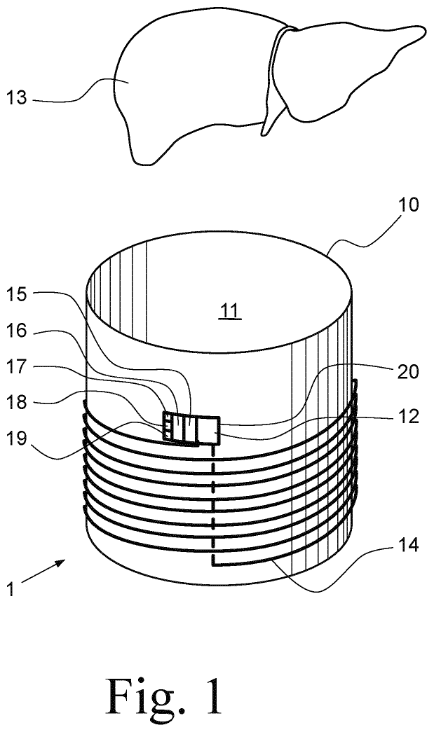

FIG. 1 illustrates schematically one exemplary embodiment of an organ container 1. The organ container 1 comprises a wall 10 surrounding an interior space 11. The interior space 11 is configured to receive an organ 13. In the present example the wall 10 is rigid. The organ 13 may be placed inside an organ bag before putting it inside the organ container 1. The organ container 1 may be closed by a lid. In one exemplary embodiment, after the organ container 1 has received the organ 13, it is placed into a cooler. One example of the cooler is an ice box. In this embodiment, the organ container 1 does not provide means for cooling the organ 13 for the duration of the transport.

The organ 13 is purposed for organ transplant. The organ 13 may be received from an organ donor. In one example the organ 13 is a synthetic organ or a synthetic tissue that may deteriorate during its transport. One example of the organ 13 is a piece of tissue. The organ 13 may be from a human donor or from any mammal. Examples of organs 13 are kidneys, pancreata, livers, intestines, hearts, or lungs.

An electric conductor 14 is connected to the wall 10, providing multiple loops around the interior space 11. The electric conductor 14 is insulated from any physical contact with the organ 13. The electric conductor 14 is not a medical electrode being in direct connection with the organ 13. Typically, medical electrodes are configured to transfer the energy of ionic currents in the organ 13 into electrical currents that can be amplified, studied, and used to help make diagnoses. The electric conductor 14 is configured to form a coil or the loop that produces an electric field. The organ 13 and its deterioration has a measurable effect on the electric field that is measured with the electric conductor 14.

In one embodiment, the electric conductor 14 forms one loop around the interior space 11. The electric conductor 14 is configured to measure an impedance value, wherein any object placed inside the loop—and in the interior space 11—affects the measured impedance. When the organ 13 is placed in the interior space 11 and surrounded by the electric conductor 14, the organ 13 has an impact on the measured impedance. In the present example the electric conductor 14 has nine loops around the interior space 11, but the number of loops is not limited. The electric conductor 14 may form any number of loops being practical for measuring the impedance. The number of loops, as well as the direction of loops may be defined by the material or the conductor, conductivity, shape of the wall 10 and the interior space 11. The wall 10 is in one embodiment made of non-conductive material, such as plastic or any other material meeting the policies for organ transplants. In one embodiment, the wall 10 is made of electrically conductive material and the electric conductor 14 is attached to the inner surface of the wall 10 while being electrically insulated from the wall 10.

In one embodiment, the electric conductor 14 is connected to the outer side of the wall 10. In one embodiment, the electric conductor 14 is connected to the inner side of the wall 10 while being insulated from any physical contact with the organ 13. In one embodiment, the electric conductor 14 is embedded in the wall 10. The electric conductor 14 is not in contact with the interior space 11 nor the organ 13 to be placed there.

In one embodiment, the organ container 1 comprises a source 15 for an electric current, that is used for measuring the impedance. In one embodiment, the source 15 is a chargeable or replaceable battery. In one embodiment, the source 15 is connectable to an external power source to provide the impedance measurement. In one embodiment, the source 15 is connectable to an external power source by a wired connection. In one embodiment, the source 15 is connectable to an external power source by a wireless, inductive connection. The inductive connection may be used to charge the battery of the source 15.

An impedance meter 16 is configured to measure the impedance value from the electric conductor 14. The impedance is measured as a resistance to alternating current. In one embodiment, the impedance meter 16 measures the current, the frequency and the voltage drop over the electric conductor 14. In one embodiment, the impedance meter 16 provides the alternating current to the electric conductor 14. In one embodiment, the impedance meter 16 is configured to control the source 15 to provide alternating current to the electric conductor 14.

The organ container 1 comprises at least one processor 17 and a memory 18 for storing instructions that, when the instructions are executed, cause the organ container 1 to perform the functionality described herein. A transceiver 19 is configured to provide a communication link from the organ container 1 to external devices, such as a control computer. The control computer 120 may reside at least partially in a cloud computing environment. In one embodiment, the processor 17 and the memory 18 are configured to control the functionality of other electronic components of the organ container 1, such as the impedance meter 16. In one embodiment, the processor 17 and the memory 18 are distributed to a system outside the organ container 1.

In one embodiment, the electronic components connectable to the electric conductor 14 are arranged into a single-body module 20. Alternatively, the electronic components may be distributed to various positions on the organ container 1. In one embodiment, the electronic components are installed to a previously manufactured organ container. In one embodiment, the electric conductor 14 and the module 20 are provided as an additional kit, that may be placed around the previously manufactured organ container. In one embodiment, the electric conductor 14 and the module 20 are arranged into a cooler box having a predefined position for the previously manufactured organ container; the electric conductor 14 may for a loop around the predefined position. The combination of the electric conductor 14, the module 20 and the previously manufactured organ container form the organ container 1 according to the present disclosure. The interior space 11 of the previously manufactured organ container acts as the interior space 11 surrounded by at least one loop of the electric conductor 14.

The processor 17 causes the organ container 1 to measure a first impedance value of the system of the electric conductor 14, the interior space 11 and any item positioned inside the interior space 11. The first impedance value is measured at a first period of time. In one embodiment, the first impedance value is measured at one frequency, wherein the frequency is selected from a range between 0.1 Hz and 1 GHz. In one embodiment, the first impedance value is measured as a frequency spectrum, wherein the frequency spectrum is selected as a range between 0.1 Hz and 1 GHz, or as multiple measurement frequencies within said range.

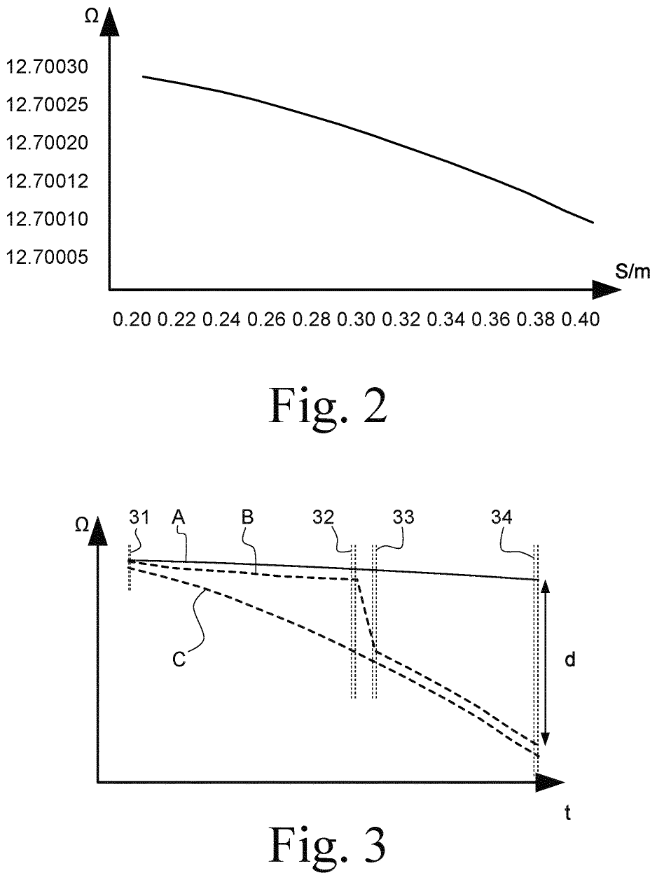

Conductance of the organ 13 has a measurable effect on the impedance of the electric conductor 14, when the organ 13 is placed inside the interior space 11. As the organ 13 deteriorates, conductance of the organ 13 increases, which causes the impedance of the coil, i.e. the loop of the electric conductor 14, near the organ 13 to decrease. One example of the relation of the measured impedance and the conductance of a deteriorating organ 13 is illustrated in an exemplary graph of FIG. 2. The graph is obtained with the coil excitation of 1 mA at 1 MHz. The Y-axis illustrates one example of the measured impedance and the X-axis the conductance of the organ in multiple stages of deterioration. Tissue-specific simulation database may be obtained from: Hasgall P A, Di Gennaro F, Baumgartner C, Neufeld E, Lloyd B, Gosselin M C, Payne D, Klingenböck A, Kuster N, “IT'IS Database for thermal and electromagnetic parameters of biological tissues,” Version 4.1, Feb. 22, 2022, DOI: 10.1309/VIP21000-04-1.

FIG. 3 illustrates three exemplary plots of the impedance measurement, in one selected frequency as a function of time. The first period of time is illustrated as a section between the dashed lines 31, when the first impedance measurement is completed. The first impedance measurement is stored to the memory 18. In one embodiment, the first impedance measurement is relayed via the transceiver 19 to a data storage outside the organ container 1. The first impedance measurement, the single measured impedance or the impedance spectrum is used to define an initial value that may be compared to later measurements. In one embodiment, the first measurement sets a base value for an organ deterioration state. The organ deterioration state may be relatable with traditional transplant diagnostics measures.

The second impedance value is measured at a second period of time. The second period of time is illustrated as a section between the dashed lines 32. The second impedance measurement method is similar to the first impedance measurement. The difference between the first impedance value and the second impedance is calculated by comparing the two values. The deterioration state of the organ 13 is defined based on said difference. The difference indicates the deterioration rate or change in the organ 13 viability; increasing difference means less viable organ 13. The deterioration state may be used to assess viability or the organ 13. The deterioration state is a relative value as each new organ 13 placed into the organ container 1 may provide different first impedance measurement values. Orientation, size, age, or other variables may cause differences to the first measurement value.

The three plots show different organ deterioration profiles, wherein plot A is illustrated by a solid line and refers to an exemplary organ A. The plot A does not show significant difference in the measured impedance between the first impedance measurement and the second impedance measurement. Plot B, referring to an exemplary organ B, shows minor difference in the measured impedances. As the difference in the measured impedances may be related to the level of deterioration in the organ, organ B may be slightly deteriorated at the time of the second measurement. Plot C, referring to an exemplary organ C, shows major difference in the measured impedances, when compared to A or B. It may be assumed that organ C is severely deteriorated at the time of the second measurement.

Throughout this disclosure, the number of measurements is not limited to “first” and “second” measurement, as the measurements may be repeated throughout the transportation. In one embodiment, the organ 13 is measured for the first time when the organ container 1 has received the organ 13 and the second time just before the organ is removed from the organ container 1. Any number of measurements may be conducted may be conducted in the meaning of the first measurement and second measurement.

Referring to FIG. 3, a third impedance value is measured at a third period of time. The third period of time is illustrated as a section between the dashed lines 33; and a fourth measurement is indicated between the dashed lines 34. The third measurement shows a sudden drop in the measured impedance of organ B. The sudden change in the measured impedance of organ B may indicate that something drastic has happened, which causes sudden deterioration of organ B.

From the fourth measurement 34 may be assumed the difference d between deteriorations of organs A, B and C. The transplanting surgeon may reject organs B and C, or subject them to more detailed examination. Organ A shows minor signs of deterioration; therefore it may be regarded as the best specimen of the three organs A, B, C. In one embodiment, the organ container 1 measures multiple measurements at multiple periods of time and detects a trend from the multiple measurements. Examples of such trends are illustrated in the plots A, B and C. The trend indicates the deterioration state of the organ 13. The viability assessment of the organ 13 may be defined based on said trend. In one embodiment, the organ container 1 is configured to detect the trend. In one embodiment, the organ container 1 exports the measured data to external processor, wherein the external processor is configured to detect said trend.

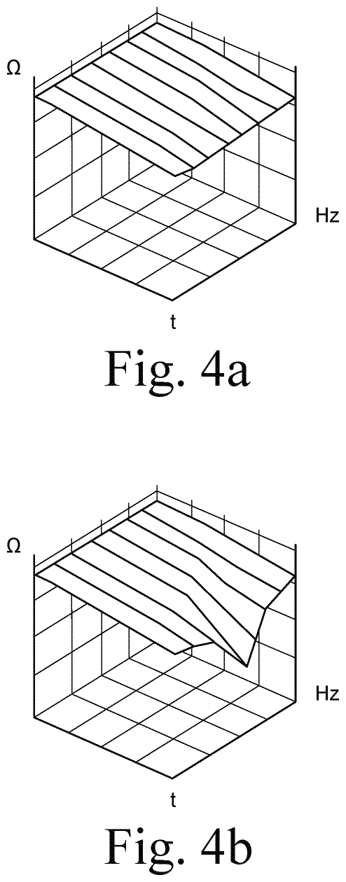

FIG. 4a and FIG. 4b illustrate schematically two examples of the impedance frequency spectrum measurements. The frequency spectrum may be measured from multiple frequencies within the operational range of the organ container electronics. In the example of FIG. 4a the deterioration has been minimal through all measurements. Alternatively, the example of FIG. 4b shows a significant dip in the frequency spectrum during the transport. The deterioration of the organ 13 may be detected only from limited frequencies. As the deterioration and/or other environmental properties are difficult to predict, the frequency affected by the organ deterioration may be shown as a change in the frequency spectrum. In one embodiment, the organ container 1 is configured to detect the trend and any change in the frequency spectrum. In one embodiment, the organ container 1 exports the measured data to external processor, wherein the external processor is configured to detect said trend.

Environmental changes may influence the measured impedance. Having multiple measurements may alleviate the problem of filtering the environmental changes from the organ deterioration. In one embodiment, the environmental changes or effects may be detected as outliers in the measured data that may be filtered from the results. In one embodiment, the filtered data is logged with timestamps, allowing the transport personnel to evaluate the cause for outlier data measurements.

In one embodiment, the organ container 1 comprises a display 12 configured to indicate the viability of the organ 13 residing in the interior space 11. In one embodiment, the processor 17 defines that the organ 13 is not viable, if the deterioration state exceeds at least one predefined limit. In one embodiment, the display 12 is part of a user interface configured into the organ container. In one embodiment, the display 12 is a simple indicator, that may illustrate by one light or colour the status of the measurements. The display 12 may indicate if the processor 17 has detected a trend in the measurements that indicates deterioration on the organ 13 being transported. In one embodiment, the display 12 illustrates the graphs of the frequency spectrum measurements, wherein the transplanting surgeon may quickly evaluate the changes occurred during the transportation.

In one embodiment, the display 12 displays raw impedance measurement values, as received from the impedance meter 16. The display 12 may show the values at a selected time, from a selected period or as differences of different measurements.

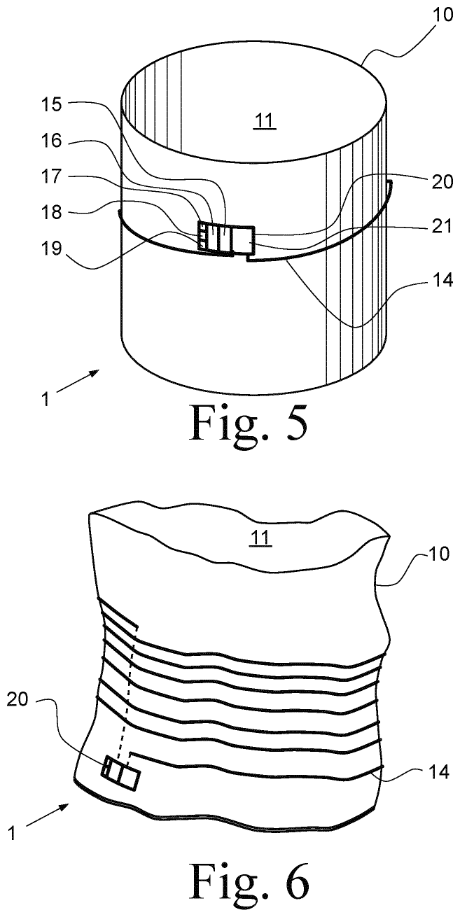

FIG. 5 illustrates one exemplary embodiment of the organ container 1 having a rigid wall 10. This embodiment comprises one loop of the electric conductor 14 surrounding the interior space 11. The impedance measurement device may comprise various components known to a skilled man.

FIG. 6 illustrates one exemplary embodiment of the organ container 1 having a non-rigid wall 10, wherein the electric conductor 14 is a flexible conductor. The organ container 1 is in one embodiment a bag, wherein the module 20 having the electronic components is placed to a position where the operation of a regular organ bag is mitigated. The electric conductor 14 is flexible, following the shapes of the bag.

The organ container 1 may be used as a regular organ container, wherein the personnel taking part in the transport of the organ 13 may follow any pre-existing policies. The regular organ container model may have either rigid or flexible walls.

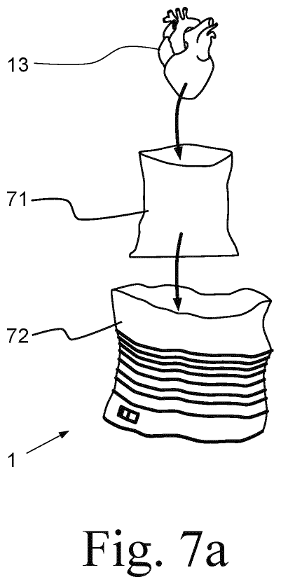

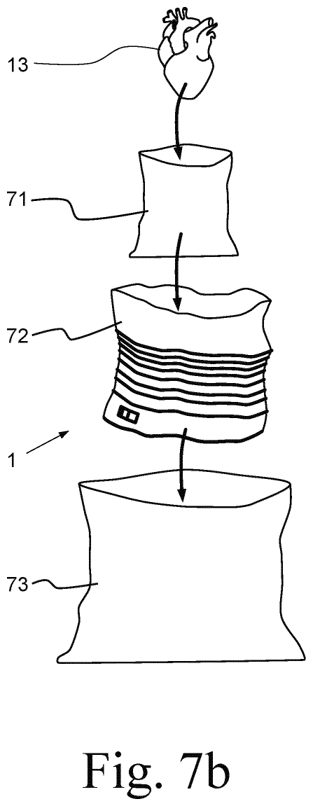

FIG. 7a illustrates one exemplary embodiment of a system, wherein the organ container 1 part of an organ container system. The organ container 1 provides one of the at least two sterile barriers 71, 72 configured to surround the organ 13. In one embodiment, the organ container 1 is a second organ bag 72, configured to surround the organ 13 positioned in a first organ bag 71. In one embodiment, the functionality of the impedance measurement is distributed over multiple components within the organ transport. In one embodiment, the organ container 1 forms a system configured to transport the organ 13.

FIG. 7b illustrates on exemplary embodiment, where the organ container 1 provides one of the at least three sterile barriers 71, 72, 73 configured to surround the organ 13, as required by some organ transport policies. The organ container 1 is in the organ container system. In this example said three sterile barriers 71, 72, 73 are configured to enclose each other. The organ container 1 may be selected to be any one of the three sterile barriers 71, 72, 73.

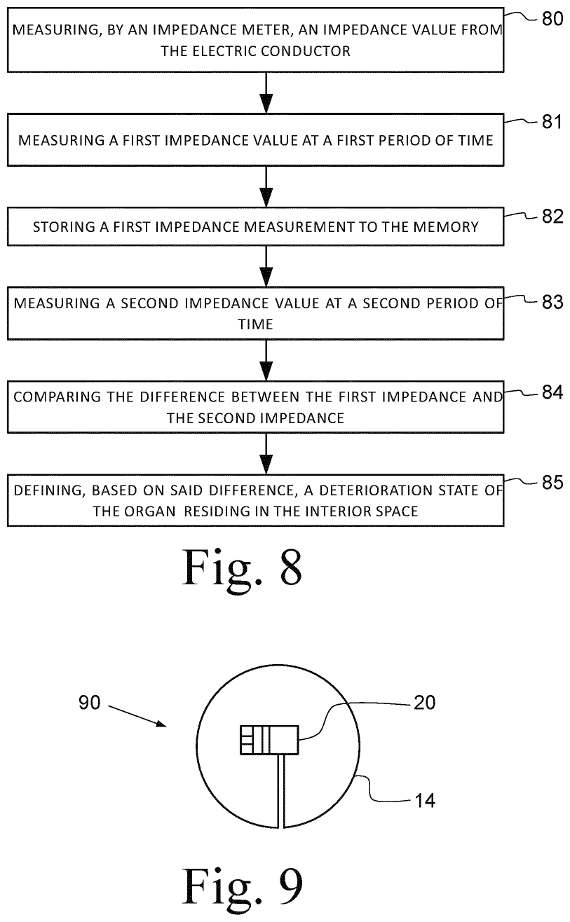

FIG. 8 illustrates a flowchart of the steps of a method of using the organ container 1 disclosed herein. Step 80 comprises measuring, by the impedance meter 16, the impedance value from the electric conductor 14. Step 81 comprises measuring the first impedance value at the first period of time. Step 82 comprises storing the first impedance measurement to the memory 18. Step 83 comprises measuring the second impedance value at the second period of time. Step 84 comprises comparing the difference between the first impedance and the second impedance. Step 85 comprises defining, based on said difference, the deterioration state of the organ 13 residing in the interior space 11.

FIG. 9 illustrates schematically one exemplary embodiment of an impedance measurement device 90 for the organ container. In this example, the impedance measurement device comprises the module 20 and the electric conductor 14. In one embodiment, the electric conductor 14 forms a loop around the module 20. In one embodiment, the module 20 is outside the loop formed by the electric conductor 14. The impedance measurement device 90 may be designed to produce the electric field towards the interior space 11. In one embodiment, the impedance measurement device comprises a shielded housing configured to limit the directions of the electric field beyond the interior space 11. The shape, size and the selection of materials may be used to provide directional measurement. In one embodiment, the impedance measurement device 90 comprises multiple loops of the electric conductor 14.

In one embodiment, the electric conductor 14 is configured to measure an impedance value, wherein any object placed in its vicinity affects the measured impedance. When the organ 13 is placed in the interior space 11 near the electric conductor 14, the organ 13 has an impact on the measured impedance.

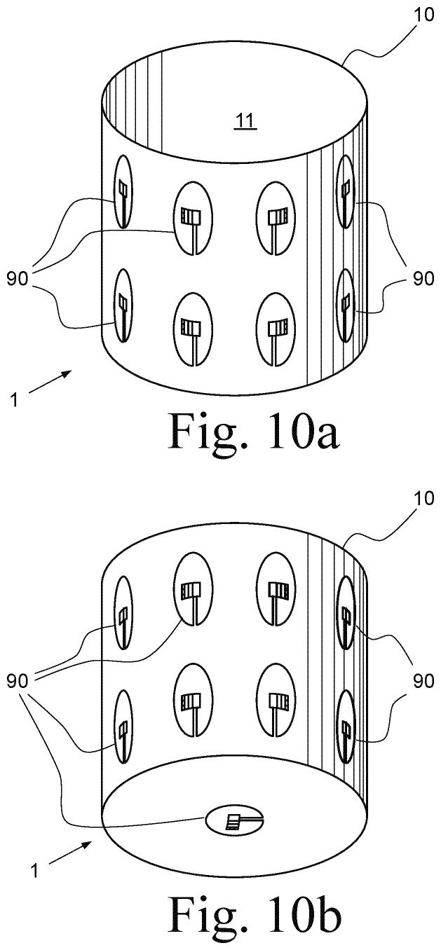

FIG. 10a and FIG. 10b illustrate schematically one exemplary embodiment of the organ container 1 from two different angles. Multiple impedance measurement devices 90 are attached to the wall 10 and to the bottom of the organ container 1. In one embodiment, the organ container 1 comprises only one impedance measurement device 90 at the bottom of the organ container 1. In one embodiment, multiple impedance measurement devices 90 provide individual measurements that may be used to calculate the deterioration of the organ 13. In one embodiment, multiple impedance measurement devices 90 may be used to detect and cancel out measurement errors. In one embodiment, placement of the multiple impedance measurement devices are used to cancel movement artifacts caused by the organ 13 moving in the organ container 1 during transport. When the impedance measurement device 90 is placed on the wall 10 or at the bottom of the organ container 1, the electric conductor 14 is forming at least one loop on the wall 10 or at the bottom, wherein an electric field caused by the impedance measurement extends into the interior space 11. In one embodiment, the multiple impedance measurement devices 90 are synchronized to provide the measurements at different times and only one impedance measurement device 90 measures the interior space 11 at a time.

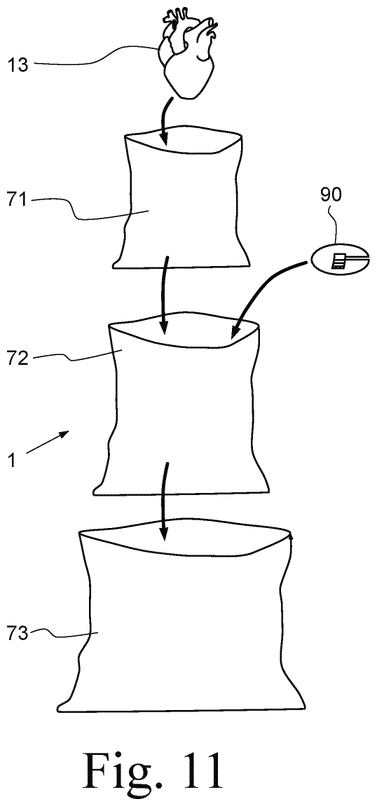

FIG. 11 illustrates one exemplary embodiment of the impedance measurement device 90, wherein the device is placed inside the organ container. In the example of FIG. 11 the impedance measurement device 90 is placed inside the second organ bag 72. In one embodiment, the impedance measurement device 90 is placed inside the organ container having rigid walls 10. The impedance measurement device comprises 10 electric conductor 14 that forms the at least one loop configured to fit in the interior space 11.

An organ container is disclosed herein. The organ container comprises a wall surrounding an interior space, wherein the interior space is configured to receive an organ; an electric conductor connected to the wall, forming at least one loop; and a source for an electric current. The electric conductor is configured to measure impedance from the interior space. An impedance meter is configured to measure impedance value from the electric conductor. At least one processor and a memory storing instructions that, when executed, cause the organ container to: measure a first impedance value at a first period of time; store a first impedance measurement to the memory; measure a second impedance value at a second period of time; compare the difference between the first impedance and the second impedance; and define, based on said difference, a deterioration state of the organ residing in the interior space. In one embodiment, the electric conductor is forming at least one loop around the interior space. In one embodiment, the electric conductor is forming at least one loop on the wall, wherein an electric field caused by the impedance measurement extends into the interior space. In one embodiment, the organ container comprises multiple loops formed by the electric conductor that face the interior space from different positions on the wall. In one embodiment, the at least one processor and the memory storing instructions that, when executed, cause the organ container to measure the impedance at a single frequency, wherein the frequency is selected between 0.1 Hz and 1 GHz. In one embodiment, the at least one processor and the memory storing instructions that, when executed, cause the organ container to measure the impedance at multiple frequencies to provide a frequency spectrum, wherein the frequency spectrum is selected as a range between 0.1 Hz and 1 GHz. In one embodiment, the at least one processor and the memory storing instructions that, when executed, cause the organ container to measure multiple measurements at multiple periods of time; to detect a trend from the multiple measurements; and to define the viability of the organ based on said trend. In one embodiment, the organ container comprises a display indicating the viability of the organ residing in the interior space. In one embodiment, the wall is a rigid wall. In one embodiment, the wall is a non-rigid wall, and the electric conductor is a flexible conductor. In one embodiment, the organ container is a part of an organ container system, and the organ container provides one of the at least two sterile barriers configured to surround the organ. In one embodiment, the organ container is a second organ bag, configured to surround the organ positioned in a first organ bag.

Alternatively, or in addition, an organ container system comprising the organ container is disclosed herein.

Alternatively, or in addition, a method for defining a deterioration state of an organ residing in an organ container is disclosed herein. Said organ container comprises: a wall surrounding an interior space, wherein the interior space is configured to receive the organ; an electric conductor connected to the wall, providing at least one loop; and a source for an electric current. The method comprises the steps of: measuring, by an impedance meter, an impedance value from the electric conductor, wherein the impedance measurement causes an electric field from the electric conductor to extend into the interior space and measure impedance from an organ residing in the interior space; measuring a first impedance value at a first period of time; storing a first impedance measurement to the memory; measuring a second impedance value at a second period of time; comparing the difference between the first impedance and the second impedance; and defining, based on said difference, the viability of the organ. In one embodiment, the method comprises measuring the impedance at a single frequency or as a frequency spectrum. In one embodiment, the method comprises measuring multiple measurements at multiple periods of time; detecting a trend from the multiple measurements; and defining the viability of the organ based on said trend. In one embodiment, the method comprises measuring the impedance at a single frequency, wherein the frequency is selected between 0.1 Hz and 1 GHz. In one embodiment, the method comprises measuring the impedance at multiple frequencies to provide a frequency spectrum, wherein the frequency spectrum is selected as a range between 0.1 Hz and 1 GHz.

Alternatively, or in addition, an impedance measurement device for an organ container is disclosed, wherein the organ container comprises a wall surrounding an interior space configured to contain an organ. The impedance meter comprises: an electric conductor forming at least one loop configured to fit in the interior space; a source for an electric current, wherein the electric conductor is configured to measure impedance value from the interior space; at least one processor and a memory storing instructions that, when executed, cause the impedance meter to: measure a first impedance value at a first period of time; store a first impedance measurement to the memory; measure a second impedance value at a second period of time; compare the difference between the first impedance and the second impedance; and define, based on said difference, a deterioration state of the organ residing in the interior space.

Alternatively, or in addition, the controlling functionality described herein can be performed, at least in part, by one or more hardware components or hardware logic components. An example of the device described hereinbefore is a computing-based device comprising one or more processors which may be microprocessors, controllers, or any other suitable type of processors for processing computer-executable instructions to control the operation of the device in order to control one or more sensors, receive sensor data and use the sensor data. The computer-executable instructions may be provided using any computer-readable media that is accessible by a computing-based device. Computer-readable media may include, for example, computer storage media such as memory and communications media. Computer storage media, such as memory, includes volatile and non-volatile, removable, and non-removable media implemented in any method or technology for storage of information such as computer-readable instructions, data structures, program modules or other data. Computer storage media includes, but is not limited to, RAM, ROM, EPROM, EEPROM, flash memory or other memory technology, CD-ROM, digital versatile disks (DVD) or other optical storage, magnetic cassettes, magnetic tape, magnetic disk storage or other magnetic storage devices, or any other non-transmission medium that can be used to store information for access by a computing device. In contrast, communication media may embody computer-readable instructions, data structures, program modules, or other data in a modulated data signal, such as a carrier wave, or other transport mechanism. As defined herein, computer storage media does not include communication media. Therefore, a computer storage medium should not be interpreted to be a propagating signal per se. Propagated signals may be present in a computer storage media, but propagated signals per se are not examples of computer storage media. Although the computer storage media is shown within the computing-based device, it will be appreciated that the storage may be distributed or located remotely and accessed via a network or other communication link, for example, by using a communication interface.

The apparatus or the device may comprise an input/output controller arranged to output display information to a display device which may be separate from or integral to the apparatus or device. The input/output controller is also arranged to receive and process input from one or more devices, such as a user input device (e.g. a mouse, keyboard, camera, microphone, or other sensor).

The methods described herein may be performed by a software in machine-readable form on a tangible storage medium e.g. in the form of a computer program comprising computer program code means adapted to perform all the steps of any of the methods described herein when the program is run on a computer and where the computer program may be embodied on a computer-readable medium. Examples of tangible storage media include computer storage devices comprising computer-readable media, such as disks, thumb drives, memory etc. and do not only include propagated signals. Propagated signals may be present in a tangible storage media, but propagated signals per se are not examples of tangible storage media. The software can be suitable for execution on a parallel processor or a serial processor such that the method steps may be carried out in any suitable order, or simultaneously.

Any range or device value given herein may be extended or altered without losing the effect sought.

Although at least a portion of the subject matter has been described in language specific to structural features and/or acts, it is to be understood that the subject matter defined in the accompanying claims is not necessarily limited to the specific features or acts described above. Rather, the specific features and acts described above are disclosed as examples of implementing the claims and other equivalent features and acts are intended to be within the scope of the claims.

It will be understood that the benefits and advantages described above may relate to one embodiment or may relate to several embodiments. The embodiments are not limited to those that solve any or all of the stated problems or those that have any or all of the stated benefits and advantages. It will further be understood that reference to ‘an’ item refers to one or more of those items.

The steps of the methods described herein may be carried out in any suitable order, or simultaneously where appropriate. Additionally, individual blocks may be deleted from any of the methods without departing from the spirit and scope of the subject matter described herein. Aspects of any of the examples described above may be combined with aspects of any of the other examples described to form further examples without losing the effect sought.

The term ‘comprising’ is used herein to mean including the method blocks or elements identified, but that such blocks or elements do not comprise an exclusive list and a method or device may contain additional blocks or elements.

It will be understood that the above description is given by way of example only and that various modifications may be made by those skilled in the art. The above specification, examples and data provide a complete description of the structure and use of exemplary embodiments. Although various embodiments have been described above with a certain degree of particularity, or with reference to one or more individual embodiments, those skilled in the art could make numerous alterations to the disclosed embodiments without departing from the spirit or scope of this specification.

1. An organ container, comprising:
a wall surrounding an interior space, wherein the interior space is configured to receive an organ;
an electric conductor connected to the wall, forming at least one loop; and
a source for an electric current,
wherein the electric conductor is configured to measure an impedance from the interior space;
an impedance meter is configured to measure impedance value from the electric conductor;
at least one processor and a memory storing instructions that, when executed, cause the organ container to:
measure a first impedance value at a first period of time;
store a first impedance measurement to the memory;
measure a second impedance value at a second period of time;
compare the difference between the first impedance value and the second impedance value; and
define, based on said difference, a deterioration state of the organ residing in the interior space.

2. An organ container according to claim 1, wherein the electric conductor is forming at least one loop around the interior space.

3. An organ container according to claim 1, wherein the electric conductor is forming at least one loop on the wall, wherein an electric field caused by the impedance measurement extends into the interior space.

4. An organ container according to claim 3, comprising multiple loops formed by the electric conductor that face the interior space from different positions on the wall.

5. An organ container according to claim 1, wherein the at least one processor and the memory storing instructions that, when executed, cause the organ container to measure the impedance at a single frequency, wherein the frequency is selected between 0.1 Hz and 1 GHz.

6. An organ container according to claim 1, wherein the at least one processor and the memory storing instructions that, when executed, cause the organ container to measure the impedance at multiple frequencies to provide a frequency spectrum, wherein the frequency spectrum is selected as a range between 0.1 Hz and 1 GHz.

7. An organ container according to claim 1, wherein the at least one processor and the memory storing instructions that, when executed, cause the organ container to measure multiple measurements at multiple periods of time; to detect a trend from the multiple measurements; and to define the viability of the organ based on said trend.

8. An organ container according to claim 1, comprising a display indicating the viability of the organ residing in the interior space.

9. An organ container according to claim 8, wherein the display displays raw impedance measurement values.

10. An organ container according to claim 1, wherein the wall is a rigid wall.

11. An organ container according to claim 1, wherein the wall is a non-rigid wall and the electric conductor is a flexible conductor.

12. (canceled)

13. (canceled)

14. (canceled)

15. A method for defining a deterioration state of an organ residing in an organ container, said organ container comprising:
a wall surrounding an interior space, wherein the interior space is configured to receive the organ;
an electric conductor connected to the wall, providing at least one loop; and
a source for an electric current,
an impedance meter, and
a memory
wherein the method comprises the steps of:
measuring, by an impedance meter, an impedance value from the electric conductor, wherein the impedance measurement causes an electric field from the electric conductor to extend into the interior space and measure impedance from an organ residing in the interior space;
measuring a first impedance value at a first period of time;
storing a first impedance measurement to the memory;
measuring a second impedance value at a second period of time;
comparing the difference between the first impedance value and the second impedance value; and
defining, based on said difference, the deterioration sate of the organ.

16. A method according to claim 15, comprising the step of measuring the impedance at a single frequency or as a frequency spectrum.

17. A method according to claim 15, comprising the step of measuring multiple measurements at multiple periods of time; detecting a trend from the multiple measurements; and defining the viability of the organ based on said trend.

18. A method according to claim 15, comprising the step of measuring the impedance at a single frequency, wherein the frequency is selected between 0.1 Hz and 1 GHz.

19. A method according to claim 15, comprising the step of measuring the impedance at multiple frequencies to provide a frequency spectrum, wherein the frequency spectrum is selected as a range between 0.1 Hz and 1 GHz.

20. An impedance measurement device for an organ container, wherein the organ container comprises a wall surrounding an interior space configured to contain an organ;
wherein the impedance measurement device comprises:
an electric conductor forming at least one loop configured to fit in the interior space;
a source for an electric current, wherein
the electric conductor is configured to measure an impedance value from the interior space;
at least one processor and a memory storing instructions that, when executed, cause the impedance meter to:
measure a first impedance value at a first period of time;
store a first impedance measurement to the memory;
measure a second impedance value at a second period of time;
compare the difference between the first impedance value and the second impedance value; and
define, based on said difference, a deterioration state of the organ residing in the interior space.
